(19) United States
(12) Patent Application Publication
(10) Pub. No.: US 2005/0119285 A1
(43) Pub. Date: Jun. 2, 2005

(54) Treatment of neurological disorders related to rapid eye movement (REM) sleep disturbances with NPY Y5 receptor antagonists

(75) Inventors: Francisca Fatima Matos, Mystic, CT (US); Jeffrey S. Sprouse, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/950,987

(22) Filed: Sep. 27, 2004

Related U.S. Application Data
(60) Provisional application No. 60/506,482, filed on Sep. 26, 2003.

(51) Int. Cl. A61K 31/52; A61K 31/4747

(52) U.S. Cl. 514/263.22; 514/278

(57) ABSTRACT

This invention relates to a method for treating and preventing neurological disorders related to rapid-eye-movement (REM) sleep disturbances in a mammal comprising administering to the mammal an amount of an NPY Y5 receptor antagonist which effectively reduces REM sleep.

### FIELD OF THE INVENTION

This invention relates to a method for treating and preventing neurological disorders related to rapid-eye-movement (REM) sleep disturbances in a mammal comprising administering to the mammal an amount of an NPY Y5 receptor antagonist which effectively reduces REM sleep. As used herein, the term REM sleep is defined as the period of sleep during which rapid eye movements are seen and the brain waves are fast and of low voltage as seen in the electroencephalogram (EEG) recording.

### BACKGROUND OF THE INVENTION

During sleep, a mammal experiences two types—REM and NREM (non-REM) sleep—defined by their morphology in the EEG. During REM sleep the brain waves are fast and of low voltage; this period of sleep is associated with rapid eye movements—hence the name—and with dreaming, involuntary muscle movements and irregular autonomic responses such as heart rate and respiration. These latter activities account for other commonly used nomenclature, for example, paradoxical or desynchronized sleep. REM sleep occurs 3-4 times during each night at 80 to 120 minute intervals with each occurrence lasting from 5 minutes to an hour. NREM sleep is also called slow wave and synchronized sleep and is characterized by slow brain waves of high voltage consisting of four stages of succeeding depth and is a period of sleep without dreaming. During NREM sleep, the autonomic activities such as heart rate and blood pressure are low and regular. In humans, about 20% of sleep is REM sleep and 80% is NREM sleep. Both REM sleep and NREM sleep are necessary for homeostasis and survival of all mammals.

Abnormalities in sleep architecture, sleep maintenance, impaired sleep continuity, sleep fragmentation, and brain wave distribution have been described in many psychiatric sleep disorders and psychiatric diseases such as depression, including major depression, unipolar depression, bipolar disorder, seasonal affective disorders, winter depression and dysthymia; premenstrual dysphoric disorder, obsessive compulsive disease, generalized anxiety, mania, panic, post-traumatic stress disorder, obesity and eating disorders including anorexia and bulimia; phobias, borderline personality, schizophrenia, dementia and cognitive dysfunction including Alzheimer type and Parkinson's disease and Parkinson's disease associated with depression, processing of emotional memory, fibromyalgia, rheumatoid arthritis and osteoarthritis, REM sleep behavior disorders, insomnia, hypersomnia, parasomnia, narcolepsy, sleep-related breathing disorders, sleep apnea, sleep walking, nocturnal enuresis, restless-leg syndrome, periodic limb movement in sleep and seizure disorders, including nocturnal seizures. Circadian rhythms related disorders are also associated with sleep disturbances including jet travel Get lag), especially between time zones, artificial light, delayed and advanced sleep phase syndrome, non-24-hour sleep-wake disorder and shift work hours may be poorly synchronized with internal circadian clocks. As a consequence of modern life schedules, performance degradation may manifest in loss of manual dexterity, reflexes, memory, winter depression, and general fatigue derived from lack of enough sleep.

Observations of major depressed patients, war related anxieties, post traumatic disorders, state of bereavement, suicidal patients with depression, schizo-affective disorder and schizophrenia, have indicated increased frequency and duration of disturbances due to REM sleep and a general reduction in slow wave states. Major depression is associated with REM sleep disturbances, in particular, disinhibition of REM sleep including shortenings of REM latency (defined as the time between sleep onset and occurrence of the first REM period) and increases in REM density and about 90% of patients with major depression have some form of sleep abnormality read in EEG. Accordingly, the majority of antidepressant drugs have been found to reduce REM sleep at therapeutic doses (Winokur) and many clinicians regard the beneficial effect of a selected antidepressant on suppressing REM sleep when making therapeutic options for treating depression in patients. The effect of antidepressant drugs on REM sleep suppression has been shown for representative agents of antidepressant mechanistic classes including tricyclic antidepressants (TCAs), monoamine oxidase inhibitors (MAOs), and selective serotonin re-uptake inhibitors (SSRI). TCAs and SSRIs have been shown to produce immediate (40-85%) and sustained (30-50%) suppression in REM sleep while the MAOs totally suppress REM sleep. Additionally, total or partial sleep deprivation or phase advance of the sleep cycle are effective treatments in patients with unipolar depression and other forms of depression including premenstrual dysphoric disorder. Therefore there is a strong correlation between sleep and manipulations sleep cycles and depression disorders. There is a clear need to continue the search for new and effective drugs for the treatment and prevention of REM sleep disorders.

Neuropeptide Y (NPY), a 36 amino acid peptide neurotransmitter, is a member of the pancreatic class of neurotransmitters/neurohormones which has been shown to be present in the central and peripheral nervous system and mediate numerous biological responses, including food intake, pain, homeostasis, seizure, anxiety, alcohol intake, endocrine responses, sleep, sedation, via NPY specific receptors (e.g. Y1, Y2, Y5 receptors). In laboratory animals, NPY has been shown to have sleep-promoting activity, shortening sleep latency, stimulate NREM sleep and modulates secretion of endocrine hormones associated with increased REM sleep. In normal humans, intravenous administration of NPY enhanced sleep period time and stage 2 sleep, reduced sleep latency and time awake and modulated REM sleep (Antonijevic et al. 2000). Therefore, agents capable of blocking NPY receptor binding and inhibiting the activity of NPY are expected to modulate sleep, including REM and NREM sleep in mammals having neurological and sleep disorders.

WO 03/051356 discloses the use of certain NPY Y5 antagonists for enhancing and improving the quality of sleep through increases in the duration or amount of REM sleep. The foregoing patents and patent applications are incorporated by reference herein in their entirety.

### SUMMARY OF THE INVENTION

The present invention provides a method of reducing REM sleep in a mammal comprising administering to a mammal an amount of an NPY Y5 antagonist, which is effective in reducing REM sleep.

In a preferred embodiment, the NPY Y5 antagonist is a compound of the formula 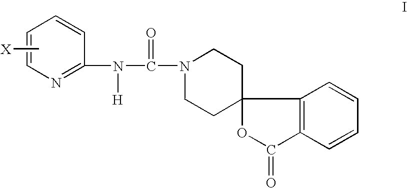
or a pharmaceutically acceptable salt, solvate or prodrug thereof or of any of the foregoing,
wherein X is selected from the group consisting of chlorine, bromine, iodine, trifluoromethyl, hydrogen, cyano, C₁ to C₆ alkyl, C₁ to C₆ alkoxy, C₅ or C₆ cycloalkyl, ester, amido, aryl, and heteroaryl.

Most preferably, the NPY Y5 antagonist of the formula I is a compound of formula 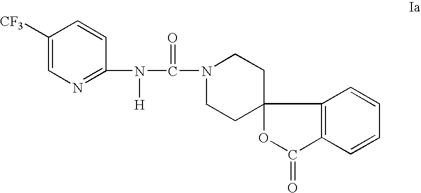
or a pharmaceutically acceptable salt, solvate or prodrug thereof or of any of the foregoing,

In another preferred embodiment, the NPY Y5 antagonist is a compound of the formula 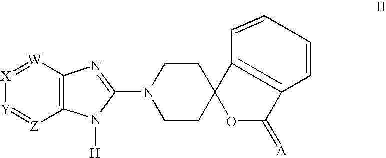
or a pharmaceutically acceptable salt, solvate or prodrug thereof or any of the foregoing; wherein A is oxygen or hydrogen;
W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono-or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono-and di(C₁-C₆)alkylamino(C₁-C6)alkyl.
or a pharmaceutically acceptable salt, solvate or prodrug thereof or of any of the foregoing. Most preferably the compound of formula II is a compound of the formula

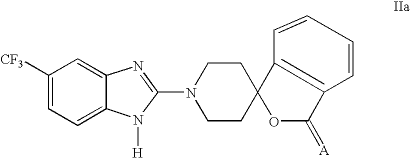

This invention provides a method of treating and preventing neurological disorders characterized by excessive rapid-eye movement (REM) sleep in mammals including humans by administering to the mammal an amount of an NPY Y5 receptor antagonist which is effective in reducing REM sleep.

Neurological disorders characterized by abnormalities and/or excessive rapid-eye movement (REM) sleep which are contemplated for treatment by the present invention include many psychiatric disorders and psychiatric diseases such as depression, including major depression, unipolar depression, bipolar disorder, seasonal affective depressive disorders, winter depression, dysthymia; premenstrual dysphoric disorder, suicidal patients with depression; obsessive compulsive disease, generalized anxiety, panic, post-traumatic stress disorder, obesity and eating disorders including anorexia and bulimia, phobias, borderline personality, schizo-affective disorder and schizophrenia, dementia and cognitive dysfunction including Alzheimer type and Parkinson's disease and Parkinson's disease associated with depression, processing of emotional memory, fibromyalgia, rheumatoid arthritis and osteoarthritis, narcolepsy, sleep-related breathing disorders, nocturnal enuresis, restless-leg syndrome, seizures and circadian rhythms related disorders including jet travel Get lag), especially between time zones. Decreases in REM latency and increases in REM density have been reported in major depression and post traumatic stress disorders, including anxieties related to war. In a preferred embodiment the disorder is a depression disorder selected from the group consisting of major depression, unipolar depression, bipolar disorder, seasonal affective depressive disorder, winter depression, dysthymia, suicidal patients with depression, Alzheimer and Parkinson's disease associated with depression.

In one embodiment of the present invention, the NPY Y5 antagonist is administered to the mammal prior to experiencing the neurological disorder.

In another embodiment, the NPY Y5 antagonist is administered to a mammal predisposed to or at risk of experiencing the neurological disorders.

This invention also provides a method for treating neurological disorders characterized by excessive REM sleep in a mammal by administering to a mammal an amount of an NPY Y5 antagonist effective in reducing REM sleep wherein the antagonist is a compound of formula 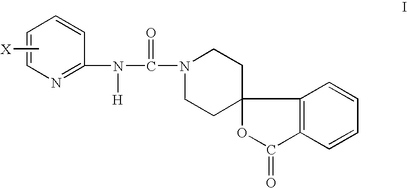
or a pharmaceutically acceptable salt, solvate or prodrug thereof or of any of the foregoing,
wherein X is selected from the group consisting of chlorine, bromine, iodine, trifluoromethyl, hydrogen, cyano, C₁ to C₆ alkyl, C₁ to C₆ alkoxy, C₅ or C₆ cycloalkyl, ester, amido, aryl, and heteroaryl.

In a preferred embodiment, the NPY Y5 antagonist is a compound of formula 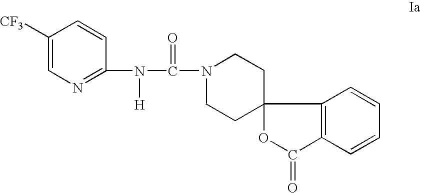
or a pharmaceutically acceptable salt, solvate or prodrug thereof or of any of the foregoing,

This invention further provides a method for treating neurological disorders characterized by excessive REM sleep in a mammal by administering to a mammal an amount of an NPY Y5 antagonist wherein the antagonist is a compound of formula 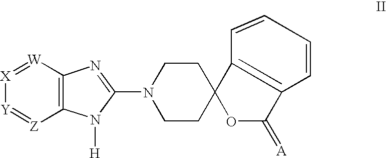
or a pharmaceutically acceptable salt, solvate or prodrug thereof or any of the foregoing; wherein A is oxygen or H₂.
W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono-or di-(C₁-C₆)alkylamino or (C₁-C₆)alkoxy, (C₃-C₇)cycloalkyl, (C₃-C₇)cyCloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono- and di(C₁-C₆)alkylamino(C₁-C₆)alkyl.

In a preferred embodiment, the NPY Y5 antagonist is a compound of the formula 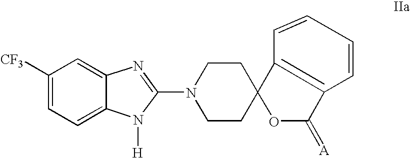
or a pharmaceutically acceptable salt, solvate or prodrug thereof or of any of the foregoing.

For compounds having asymmetric centers, all optical isomers, racemates and mixtures thereof are encompassed in the present invention.

Where a compound exists in various tautomeric forms, the invention is not limited to any one of the specific tautomers.

The present invention further provides a pharmaceutical composition comprising a compound or modulator as described above in combination with a physiologically acceptable carrier or excipient.

In one embodiment of the above-cited method, the NPY Y5 antagonist is administered to the mammal prior to experiencing REM sleep disorder.

In another embodiment of the above-cited method, the NPY Y5 antagonist is administered to a mammal predisposed to or at risk of experiencing REM sleep disorders.

The present invention provides a method of modulating REM sleep which comprises decreasing the rate of eye movement, reducing the density and latency of REM sleep, disrupting REM sleep and increasing non-REM sleep and total sleep consolidation.

In another embodiment the present invention provides a method of reducing REM sleep in a dose-related manner in a mammal which comprises administering to the mammal an amount of an NPY Y5 antagonist of formula I or 11 which is effective in reducing REM sleep.

“Latency of REM” as used herein refers to time from first occurrence of stage 2 sleep to first occurrence of REM sleep.

The term “density of REM” as used herein refers to number of REM movements per time and the amount of time spent in REM sleep.

The term “sleep latency” as used herein refers to time from lights out or ‘falling asleep’ to first occurrence of stage 2 sleep.

The term “disruption of REM sleep” as used refers to any situation that adversely interferences with a normal REM latency and density.

The term “consolidation of sleep” as used herein refers to bouts of sleep throughout the 24-hour day: roughly every 20 minutes, a laboratory animal completes a sleep/wake cycle while a human consolidate sleep into a single period per day, normally interrupted by only very short bouts of wakefulness.

### DETAILED DESCRIPTION OF THE INVENTION

The compounds of Formula I and Formula II can be prepared by the synthetic methods described and referred to in WO 02/48152 which is hereby incorporated by reference-herein in its entirety.

Representative compounds of Formula I include, but are not limited to:
1′-(4-t-butyl-pyridylcarbamoyl)-spiroisobenzofuran-1,4′-piperidine-3-one;
1′-(4-isopropyl-pyridylcarbamoyl)-spiroisobenzofuran-1,4′-piperidine-3-one;
1′-(4-trifluoromethyl-pyridylcarbamoyl)-spiroisobenzofuran-1,4′-piperdine-3-one;

Representative compounds of Formula II include but are not limited to
1′-(1H-benzimidazol-2-yl)-spiro[isobenzofuran-1,4′-piperidin]-3-one;
1′-(5-cyano-1H-benzimidazol-2-yl)-spiro[isobenzofuran-1,4′-piperidin]-3-one;
1′-(5-acetyl-1H-benzimidazol-2-yl )-spiro[isobenzofuran-1,4′-piperidin]-3-one;
1′-(5-carboxy-1H-benzimidazol-2-yl )-spiro[isobenzofuran-1,4′-piperidin]-3-one methyl ester;
1′-(5′-pyridin-3-yl-1H-benzimidazol-2-yl)-spiro[isobenzofuran-1,4′-piperidin]-3-one;
1′-(5-methyl-1H-benzimidazol-2-yl )-spiro[isobenzofuran-1,4′-piperidin]-3-one;
1′-(5-methoxy-1H-benzimidazol-2-yl)-spiro[isobenzofuran-1,4′-piperidin]-3-one;
1′-(5-chloro-1H-benzimidazol-2-yl )-spiro[isobenzofuran-1,4′-piperidin]-3-one;
1′-(5-fluoro-1H-benzimidazol-2-yl)-spiro[isobenzofuran-1,4′-piperidin]-3-one; and
1′-(5-trifluoromethyl-1H-benzimidazol-2-yl)-spiro[isobenzofuran-1,4′-piperidin]-3-one;

Representative compounds of Formula II include, but are not limited to,
(1) ′-(6-trifluoromethyl-3-H-imidazo[4,5-b]pyridine-2-yl)-spiro[isobenzofuran-1,4′-piperidin]-3-one;
1′-(7-chloro-1H-benzimidazol-2-yl)-spiro[isobenzofuran-1,4′-piperidin]-3-one;
1′-(1H-benzimidazol-2-yl)-spiro[isobenzofuran-1,4′-piperidin]-3-one;
1′-(5-n-propylsulfonyl-1H-benzimidazol-2-yl)-spiro[isobenzofuram-1,4′-piperidin]-3-one;
1′-(5 cyano-1H-benzimidazol-2-yl)-spiro[isobenzofuran-1,4′-piperidin]-3-one;
1′-(5-acetyl-1-H-benzimidazol-2-yl)-spiro[isobenzofuran-1,4′-piperidin]-3-one;
1′-(5-carboxy-1H-benzimidazol-2-yl)-spiro[isobenzofuran-1,4′-piperidin]-3-one, methyl ester;
1′-(5′pyrazin-2-yl-1H-benzimidazol-2-yl)-spiro[isobenzofuran-1,4′-piperidin]-3-one;
′-(5′pyridin-3-yl-1H-benzimidazol-2-yl)-spiro[isobenzofuran-1,4′-piperidin]-3-one;
1′-(5-trifluorometoxy-1H-benzimidazol-2-yl)-spiro[isobenzofuran-1,4′-piperidin]-3-one;
1′-(5-methyl-1H-benzimidazol-2-yl)-spiro[isobenzofuran-1,4′-piperidin]-3-one;
1′-(5-benzoyl-1H-benzimidazol-2-yl)spiro[isobenzofuran-1,4′-piperidin]-3-one;
1′-(5-methoxy-1H-benzimidazol-2-yl)-spiro[isobenzofuran-1,4′-piperidin]-3-one;
1′-(5-chloro-1H-benzimidazol-2-yl )-spiro[isobenzofuran-1,4′-piperidin]-3-one;
6-bromo-7-chloro-2-(spiro[isobenzofuran-1,4′-piperidin]-3-one-3H-imidazo[4,5-b]pyridine;
1′-(5-fluoro-1H-benzimidazol-2-yl)-spiro[isobenzofuran-1,4′-piperidin]-3-one;
1′-(5-methyl-1H-benzimidazol-2-yl)-spiro[isobenzofuran-1,4′-piperidin]-3-one;
1′-(5-methylsulfonyl-1H-benzimidazol-2-yl)-spiro[isobenzofuran-1,4′-piperidin]-3-one;
1′-(5-oxazol-2-yl-1H-benzimidazol-2-yl )-spiro[isobenzofuran-1,4′-piperidin]-3-one;
1′-(5,6-difluoro-1H-benzimidazol-2-yl)-spiro[isobenzofuran-1,4′-piperidin]-3-one;
1′-(5phenyl-1H-imidazo[4,5-b]pyrazin-2-yl )-spiro[isobenzofuran-1,4′-piperidin]-3-one;
1′-(5-trifluoromethyl-1H-benzimidazol-2-yl)-spiro[isobenzofuran-1,4′-piperidin]-3-one;
1′-(5,7-dichloro-1H-benzimidazol-2-yl)-spiro[isobenzofuran-1,4′-piperidin]-3-one;
1′-(5,6-dimethoxy-1H-benzimidazol-2-yl)-spiro[isobenzofuran-1,4′-piperidin]-3-one;
1′-(5-trifluoromethylsulfonyl-1H-benzimidazol-2-yl)-spiro[isobenzofuran-1,4′-piperidin]-3-one;
1′-(5-(3,5-dimelthyl-isoxazol-4-yl )-1H-benzimidazol-2-yl)-spiro[isobenzofuran-1,4′-piperidin]-3-one;
1′-(5-ethoxy-1H-benzimidazol-2-yl )-spiro[isobenzofuran-1,4′-piporidin]-3-one; and
5-chloro-2-(spiro[isobenzofuran-1,4′-piperidin]-3-one-3H-imidazo[4,5-b]pyridine.

The compounds of Formula I and II which are basic in nature are capable of forming a wide variety of different salts with various inorganic and organic acids. Although such salts must be pharmaceutically acceptable for administration to animals, it is often desirable in practice to initially isolate a compound of the Formula I and 11 from the reaction mixture as a pharmaceutically unacceptable salt and then simply convert the later back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, e.g. salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate, i.e., 1,1′-methylene-bis-(2-hydroxy-3-naphthoate), salts.

The compounds of Formula I and 11 may advantageously be used in conjunction with one or more other therapeutic agents, for instance, different antidepressant agents such as tricyclic antidepressants (e.g. amitriptyline, dothiepin, doxepin, trimipramine, butripyline, clomipramine, desipramine, imipramine, iprindole, lofepramine, nortriptyline or protriptyline), monoamine oxidase inhibitors (e.g. isocarboxazid, phenelzine or tranylcyclopramine) or 5-HT re-uptake inhibitors (e.g. fluvoxamine, sertraline, fluoxetine or paroxetine), and/or with antiparkinsonian agents such as dopaminergic antiparkinsonian agents (e.g. levodopa, preferably in combination with a peripheral decarboxylase inhibitor e.g., benserazide or carbidopa, or with a dopamine agonist e.g., bromocriptine, lysuride or pergolide). It may also be used with acetocholinesterases such as donepezil. It is to be understood that the present invention covers the use of a compound of Formula I and 11 or a physiologically acceptable salt or solvate thereof in combination with one or more other therapeutic agents.

Biological activity of the NPY Y5 antagonist compounds of the present invention was determined in vivo sleep studies in laboratory experiments described herein below. Results presented herein showed that the NPY Y5 receptor antagonists of formula la and IIa affected sleep (REM and NREM) in a laboratory animal while the NPY Y1 antagonist had only slight effects on sleep variables.

The compounds of the invention are generally administered as pharmaceutical compositions in which the active principle is mixed with a pharmaceutical excipient or carrier. The active compound or principle may be formulated for oral, buccal, intramuscular, parenteral (e.g. intravenous, intramuscular or subcutaneous) or rectal administration or in a form suitable for administration by inhalation or insufflation.

Suitable forms of oral administration include tablets, capsules, powders, granules and oral solutions or suspensions, sublingual and buccal forms of administration.

When a solid composition is prepared in tablet form, the main excipient is mixed with a pharmaceutical excipient such as gelatin, starch, lactose, magnesium stearate, talc or gem arabic. Tablets may be coated with a suitable substance like sugar so that a given quantity of the active compound is released over a prolonged period of time.

Liquid preparations for oral administration may be in the form of a solution, syrup, or suspension. Such liquids may be prepared by conventional methods using pharmaceutically acceptable ingredients such as suspending agents (e.g. sorbitol syrup); emulsifying agents (e.g. lecithin); non-aqueous vehicles (e.g. ethyl alcohol); and preservatives (e.g. sorbic acid).

Formulations for parenteral administration by injection or a infusion may be presented in unit dosage form e.g. in ampules in the form of solutions or emulsions in oily or aqueous vehicles.

The compositions may also be formulated in rectal formulations such as suppositories or retention enemas.

For intranasal or inhalation administration, the compounds are delivered in the form of a solution or suspension from a pump spray or a container pressurized with suitable propellant.

In connection with the use of compounds of Formulas I or II it is to be noted that these compounds may be administered either alone or in combination with a pharmaceutically acceptable carrier. Such administration may be carried out in single or multiple doses. More particularly the composition may be combined with various pharmaceutically acceptable inert carriers in the form of tablets, capsules, lozenges, hard candies, powders, syrup, aqueous suspension, injectable solutions, elixirs, syrups, and the like.

A proposed dose of the active compounds of the invention for oral, parenteral or buccal administration to the average adult human for the treatment of the conditions referred to above (e.g. depression) is about 0.1 to about 200 mg of the active ingredient per unit dose which could be administered, for example, 1 to 4 times per day.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

This invention is based upon the discovery that NPY Y5 antagonists can suppress REM sleep. Accordingly, this invention provides a method of treating and preventing sleep disorders characterized by REM in a mammal, which method comprises administering to the mammal an amount of an NPY Y5 antagonist effective in treating and preventing REM sleep disorders.

The present invention also provides a method for treating and preventing REM sleep disorders in a mammal by administering to the mammal therapeutically effective amount of an NPY Y5 antagonist wherein the NPY Y5 antagonist are compounds of the formula la and IIa.

The present invention also provides a method for treating and preventing REM sleep disorders in a mammal by administering to the mammal therapeutically effective amount of an NPY Y5 antagonist wherein the NPY Y5 antagonist are compounds of the formula la and IIa.

### EXAMPLES

Considerations of rat and human sleep study: Rat sleep and human sleep have all of the necessary fundamental similarities to permit the rat to be used as a model. First, all compounds that are hypnotics in human have hypnotic effects in rats, and all compounds that are hypnotics in rats have hypnotic effects in humans. Second, both rats and humans exhibit robust circadian modulation of sleep tendency. Third, the “homeostatic” control of sleep shares the fundamental similarity in that loss of sleep increases the amount of low-frequency EEG (“delta waves”) during subsequent compensatory NREM sleep. That is, the “depth” of sleep is characterized by the abundance of slow-wave sleep. The depth of sleep sub serves “sleep continuity” or sleep consolidation, which is the principal determinant of sleep quality. In the context of the latter, it has been argued that and higher-amplitude EEG slow-waves in NREM sleep reflects an “intensity” function of NREM because slow-wave activity in NREM sleep increases as a function of prior wake duration and is a concomitant of sleep consolidation during normal baseline sleep. Fourth, in both rats and humans, all hypnotics affect NREM sleep by decreasing the latency to sleep onset, increasing sleep time, increasing sleep depth and/or consolidation, or some combination of these effects. Fifth, during behavioral sleep, NREM and REM sleep alternate in what may be called the NREM-REM cycle. In both rats and humans, the proportion of time spent in NREM versus REM is about 4:1, and NREM sleep always precedes REM (that is, REM normally does not occur at sleep onset). Sixth, most hypnotics reduce REM sleep to some degree, and several classes of hypnotics strongly suppress REM sleep. Although the relevance is debated, REM-suppression is generally considered desirable in the case of antidepressants. Further, the relative effect of all classes of hypnotics on REM sleep is similar in rats and humans.

There are two principal differences in rat and human sleep. First, rats are night-active, whereas humans are day-active. Although striking, this difference probably has no importance per se for testing hypnotic drug effects. It is important, however, that for either species, the timing of the dose relative to the normal sleep period be taken into account when judging hypnotic efficacy. The second difference is sleep-bout length, or what we call “sleep continuity.” Humans consolidate sleep into a single period per day, normally interrupted by only very short bouts of wakefulness. Rats have bouts of sleep throughout the 24-hour day: roughly every 20 minutes, a rat completes a sleep/wake cycle. During the night (when the rat is active), sleep occupies about ⅓ of each 20-minute cycle, and REM sleep is rare. During the day (lights-on), the rat sleeps about ⅔ of each 20-minute cycle. Sleep bout-length is an extraordinarily sensitive measure of physiological sleepiness and is an important pre-clinical predictor of soporific efficacy in humans.

Sleep measurement by EEG: For the EEG sleep measurements, adult, male Wistar rats were anesthetized and surgically implanted with a cranial implant for chronic electro-encephalogram (EEG) and electromyogram (EMG) recording. At least three weeks were allowed for the animal to recover from surgery. Food and water were available ad libitum and the ambient temperature was 24±1° C. A 24-hr light-dark cycle (LD 12:12) was maintained throughout the study using fluorescent light. Light intensity averaged 35-40 lux at mid-level inside the cage. Animals were undisturbed for two days before and after each treatment. Sleep and wakefulness were determined using a microcomputer-based sleep-wake and physiological monitoring system. The system monitored amplified EEG (×10,000, bandpass 1-30 Hz; digitization rate 100 Hz, integrated EMG (bandpass 10-100 Hz, RMS integration), and telemetered body temperature and non-specific locomotor activity and drinking activity, from 16 rodents simultaneously. Arousal states were classified on-line as NREM sleep, REM sleep, wake, or theta-domihated wake every 10 seconds using EEG period and amplitude feature extraction and ranked membership algorithms. Individually taught EEG-arousal-state templates and EMG criteria differentiated REM sleep from theta-dominated wake. Drinking and locomotor activity were automatically recorded as discrete events every 10 seconds, and body temperature was recorded each minute. Data quality was assured by frequent on-line inspection of the EEG and EMG signals.

Drug Treatment: A NPY Y1 receptor antagonist was administered at 5, 10, 20 or 40 mg/kg in 0.25% methylcellulose vehicle. The NPY Y5 receptor antagonist of Formula la was administered at 5, 10 or 40 mg/kg and the NPY Y5 receptor antagonist of Formula IIa was administered at 10 and 40 mg/kg (both in 32% hydroxypropyl-betacyclodextrin vehicle). Drugs and vehicles were administered by oral gavage. Rats were randomly assigned to receive treatments in parallel groups. The recording duration for the bioassay was 30 hours before and after treatment. At least 7 days “washout” elapsed between each treatment.

Variables recorded by EEG sleep-wake variables included NREM, REM, total sleep, and duration of sleep and wake bouts and were defined and computed as follows:
Wakefulness, NREM sleep, and REM sleep: percent time in state per hour or per 5 minute bin.
Cumulation of total sleep, NREM sleep, REM sleep, locomotor activity, and drink activity: post-treatment accumulated change over baseline. Change—from-baseline scores were computed by subtracting from the post-treatment value the baseline value at the corresponding circadian time. The change-from-baseline scores were then cumulated in hourly bins, and these values were plotted.
Sleep, wakefulness, and REM sleep bouts: The longest bout and the average bout of uninterrupted sleep each hour, measured in minutes. “Interruption” is defined as 3 or more consecutive 10 sec epochs of wakefulness. An analogous quantification is carried out for bouts of wakefulness and REM sleep. Sleep bout length is of interest because it may parallel the human tendency to awaken periodically through the night (such awakenings are normally not recalled), which in turn has been shown to be an important factor determining the restorative value of sleep in humans. Pre-clinical measures of sleep bout length are also strong predictors of soporific efficacy in humans.
Locomotor activity: counts per hour or counts per 5 minute bin.
Locomotor activity intensity: locomotor activity counts per minute of EEG-defined wakefulness. This variate allows an assessment of locomotor activity that is independent of the amount of time awake, thus, it may be used to quantify the specificity of a wake- or sleep-promoting effect (Edgar et al. 1997).

Statistical Analysis—Mixed Model: Treatment effects were analyzed by a mixed model for repeated measures data. Mixed models were performed comparing each active-treatment with vehicle. For all models analysis was based on post-treatment hours with each hour adjusted for the corresponding baseline hour. Adjusting for baseline takes into account any differences between groups during baseline. The mixed model includes the fixed effects of HOUR, TREATMENT, and TREATMENT×HOUR interaction; RATS were treated as random effects. A heterogeneous autoregressive covariance structure was modeled. This covariance structure is unique to repeated measures in which variance changes over time and measurements taken closer in time are more highly correlated than those taken further apart.

Results: The NPY Y5 receptor antagonist of formula la (5, 10 and 40 mg/kg) significantly reduced REM sleep in a dose-related manner and increased NPEM sleep and sleep continuity (sleep bout length). After 40 mg/kg, REM sleep inhibiting and NREM sleep promoting effects persisted for at least 48 hours, and were still observed at 4.5 days after dosing. The extremely long duration of action observed for this compound appeared to correlate with drug exposure. The NPY Y5 receptor antagonist of formula IIa (10 and 40 mg/kg) dose dependently significantly inhibited REM sleep. A NPY Y1 receptor antagonist tested at 5, 10, 20 and 40 mg/kg, had only slight effects on sleep variables (Table 1).

TABLE 1

| | | Maximal change in REM sleep |
|---|---|---|
| Treatment | Dose | (minutes) |
| Formula Ia | 5 | −23.8* |
| | 10 | −42.6* |
| | 40 | −74.4* |
| Formula IIa | 10 | −10.5 |
| | 40 | −19.0* |

“Maximal change in REM sleep” is the cumulative time spent in REM sleep compared to vehicle controls during the first 24 hours after the drug dose. Negative values indicate a decrease or an inhibition of REM sleep in minutes. All values indicated by * are statistically different at p < 0.025 (mixed model for repeated measures).

1. A method of reducing REM sleep in a mammal which comprises administering to the mammal an amount of an NPY Y5 antagonist, which amount is effective in reducing REM sleep.

2. A method according to claim 1, wherein the NPY Y5 antagonist is a compound of the formula 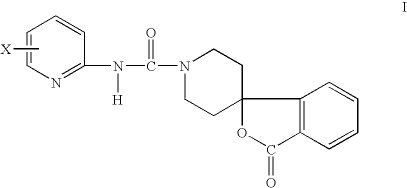
or a pharmaceutically acceptable salt, solvate or prodrug thereof or any of the foregoing; wherein X is selected from the group consisting of chlorine, bromine, fluorine, iodine, trifluoromethyl, hydrogen, cyano, C₁ to C₆ alkyl, C₁ to C6 alkoxy, C₅ or C₆ cycloalkyl; ester, amido, aryl and heteroaryl.

3. A method according to claim 1 wherein the NPY Y5 antagonist is a compound of the formula 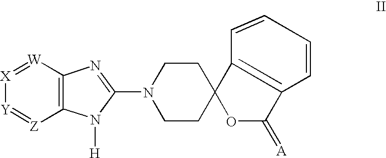
or a pharmaceutically acceptable salt, solvate or prodrug thereof or any of the foregoing; wherein A is oxygen or hydrogen, W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono-or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono-and di(C₁-C₆)alkylamino(C₁-C₆)alkyl.

4. A method of treating and preventing neurological disorders characterized by excessive rapid-eye-movement (REM) sleep in a mammal, which method comprises administering to the mammal amount of an NPY Y5 antagonist, which amount is effective in reducing REM sleep.

5. A method according to claim 6, wherein the neurological disorder is selected from the group of psychiatric diseases consisting of depression, premenstrual dysphoric disorder, obsessive compulsive disease, generalized anxiety, panic, post-traumatic stress disorder, obesity and eating disorders including anorexia and bulimia, phobias, borderline personality, schizo-affective disorder and schizophrenia, dementia and cognitive dysfunction including processing of emotional memory, fibromyalgia, rheumatoid arthritis and osteoarthritis, insomnia, hypersomnia, parasomnia, narcolepsy, sleep-related breathing disorders, nocturnal enuresis, restless-leg syndrome, seizure disorders and circadian rhythms related disorders including jet travel Get lag), especially between time zones.

6. A method according to claim 8, wherein the depression disorder is selected from the group consisting of major depression, unipolar depression, bipolar disorder, seasonal affective depressive disorders, winter depression, dysthymia, suicidal patients with depression, Alzheimer and Parkinson's disease associated with depression.

7. A method according to claim 6, wherein the NPY Y5 antagonist is administered to the mammal prior to experiencing the neurological disorder.

8. A method according to claim 6, wherein the NPY Y5 antagonist is a compound of formula 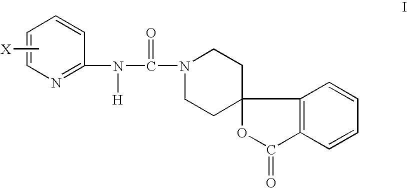
or a pharmaceutically acceptable salt, solvate or prodrug thereof or of any of the foregoing.
wherein X is selected from the group consisting of chlorine, bromine, iodine, trifluoromethyl, hydrogen, cyano, C₁ to C₆ alkyl, C₁ to C₆ alkoxy, C₅ or C₆ cycloalkyl, ester, amido, aryl, and heteroaryl.

9. A method according to claim 6 wherein the NPY Y5 antagonist is a compound of formula 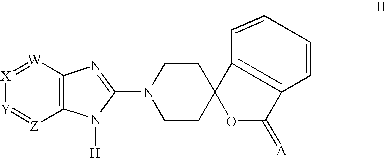
or a pharmaceutically acceptable salt, solvate or prodrug thereof or any of the foregoing;
wherein A is oxygen or hydrogen;
W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono-or di-(C₁-C₆)alkylamino or (C₁-C₆)alkoxy, (C₃-C₇)cycloalkyl, (C₃-C₇)cyCloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono-and di(C₁-C₆)alkylamino(C₁-C₆)alkyl.

10. A method according to claim 13 wherein the NPY Y5 antagonist is a compound of the formula

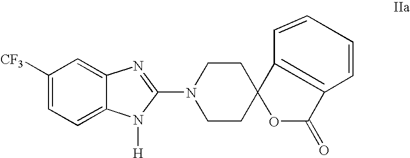

11. A method of modulating REM sleep which comprises decreasing the rate of eye movement, reducing the density and latency of REM sleep, disrupting REM sleep and increasing non-REM sleep and total sleep consolidation.
